(19) United States
(12) Patent Application Publication
(10) Pub. No.: US 2025/0157726 A1
(43) Pub. Date: May 15, 2025

(54) HYSTERETANCE COMPONENT AND APPLICATION METHOD THEREOF

(71) Applicant: SOUTHEAST UNIVERSITY, Nanjing (CN)

(72) Inventors: Ming CHENG, Nanjing (CN); Wei QIN, Nanjing (CN); Chengbo LI, Nanjing (CN); Zheng WANG, Nanjing (CN); Wei HUA, Nanjing (CN); Xinkai ZHU, Nanjing (CN)

(21) Appl. No.: 19/021,098

(22) Filed: Jan. 14, 2025

Related U.S. Application Data
(63) Continuation of application No. PCT/CN2023/128352, filed on Oct. 31, 2023.

(30) Foreign Application Priority Data
Oct. 23, 2023 (CN) 202311372958.9

(51) Int. Cl. H01F 27/34; G01R 33/14; H01F 41/04

(52) U.S. Cl. CPC H01F 27/341; G01R 33/14; H01F 41/04; H01F 2027/348

(57) ABSTRACT

The present disclosure relates to a hysteretance component, which is designed based on its definition, calculation formulas, and port characteristics. By increasing or decreasing hysteretance components in a magnetic circuit, the intensity and effect magnitude of magnetic hysteresis in a vector magnetic circuit can be estimated and controlled from the perspective of magnetic circuit, allowing the vector state of a magnetic flux to be consistent with the desired state. Based on this, an application method is proposed, involving that a target magnetic circuit is formed by connecting reluctance, magductance, and hysteretance components in series, and magnetic circuit parameters of the three components are utilized to quantitatively express magnetization, eddy current, and magnetic hysteresis phenomena, enabling technicians to selectively alter the operating characteristics of the magnetic circuit, vector magnetic quantities, and power of the magnetic circuit by adjusting the parameters.

### CROSS-REFERENCE TO RELATED APPLICATIONS

This application is a continuation of PCT/CN2023/128352, filed on Oct. 31, 2023 and claims priority of Chinese Patent Application No. 202311372958.9, filed on Oct. 23, 2023, the entire contents of which are incorporated herein by reference.

### TECHNICAL FIELD

The present disclosure relates to a hysteretance component and an application method thereof, falling within the technical field of magnetic circuit design.

### BACKGROUND

Magnetic hysteresis and loss characteristics represent two major features inherent to magnetic materials. Currently, the hysteresis characteristics of different magnetic materials are primarily characterized by hysteresis loops. The overwhelming majority of electrical apparatuses contain components formed by magnetic materials, such as iron cores of generators and power transformer in power systems, and inductance coils in electronic circuits. Therefore, it is of great significance in studying the magnetic hysteresis and loss characteristics of ferromagnetic materials in engineering.

Without considering eddy currents, macroscopically, a phase difference always presents between the magnetic induction intensity B and the magnetic field intensity H during the remagnetization of magnetic materials, known as magnetic hysteresis. To enable a more precise magnetic circuit formed by magnetic materials and simulate various steady states and transient states (such as direct current magnetic bias, voltage reduction, remanence calculation, and ferromagnetic resonance) in actual magnetic circuits, various magnetic core hysteresis models considering magnetic hysteresis are provided in the prior art. Based on the magnetization of ferromagnetic materials, hysteresis models can be classified into four types: (1) hysteresis modeling method, which is derived from micromagnetic theory, with clear physical meaning but complicated calculation, unsuitable for macroscopic applications; (2) a macroscopic magnetization theory, which is based on phenomenological principles and pure mathematical modeling, with the Preisach model proposed by German physicist F. Preisach being more classic, but which considers neither the physical basis of magnetization nor the source of magnetic hysteresis; (3) mesoscopic hysteresis theory, with the Jiles-Atherton (J-A) hysteresis model proposed by physicists D. C. Jiles and D. L. Atherton being notable, which combines the microstructure and macroscopic characterization of ferromagnetic materials to represent the properties of the ferromagnetic materials, but requires substantial experimental data to identify and solve the parameters of the J-A hysteresis model; and (4) neural network hysteresis model, which exhibits high flexibility and accuracy in simulating hysteresis behavior, but requires substantial data and computational resources and is difficult to elucidate the generation mechanism of magnetic hysteresis.

In summary, current research on magnetic materials still lays emphasis on achieving accurate and efficient simulation and analysis of magnetic hysteresis and loss characteristics. However, there is a lack of research and solutions in response to estimating and controlling, from the perspective of magnetic circuits, the intensity of magnetic hysteresis in magnetic materials and the effects caused by magnetic hysteresis such as magnetic hysteresis loss and remanence effects.

### SUMMARY

The present disclosure provides a hysteretance component, which is designed based on various properties, and applied to magnetic circuits to change the intensity of magnetic hysteresis existing in the magnetic circuits and control the magnitude of effects caused by the magnetic hysteresis.

To solve the above technical problems, the present disclosure employs the following technical solution: a hysteretance component has a hysteretance value

$$C = \frac{μ}{ω sin γ} \frac{A}{h} = \frac{κ A}{h}$$

determined based on a length h, a cross-sectional area A, a magnetic conductivity μ, and a magnetic hysteresis angle γ of the hysteretance component, and based on an angular frequency ω of a magnetic source in a magnetic circuit where the hysteretance component is located, with the unit of Wb·s/A=Ω·s². The physical meaning of the hysteretance value C is a ratio of an integral of a magnetic flux Φ_{C} flowing through the hysteretance component over time to magnetomotive forces [symbol] _{C} across two terminals of the hysteretance component, i.e.,

$$C = - \frac{\int {Φ}_{C} dt}{{ℱ}_{C}} ,$$

κ representing a magnetic medium coefficient,

$$κ = \frac{μ}{ω sin γ} ,$$

and—representing that a reference direction of the magnetomotive force [symbol] _{C} across the two terminals of the hysteretance component and a reference direction of the magnetic flux Φ_{C} flowing through the hysteretance component are opposite to preset reference directions.

For a structure of a hysteretance component including at least two sub-hysteretance components, in a case that n sub-hysteretance components are connected in series, the hysteretance value of the overall series connection structure is

$$C = 1 / ( \frac{1}{{C}_{1}} + \frac{1}{{C}_{2}} + … \frac{1}{{C}_{n - 1}} + \frac{1}{{C}_{n}} ) ,$$

and in a case that n sub-hysteretance components are connected in parallel, a hysteretance value of the overall parallel connection structure is C=C₁+C₂ . . . +Cₙ₋₁+Cₙ.

In a preferred technical solution of the present disclosure, if environmental variables of the magnetic circuit where the hysteretance component is located change over time, the hysteretance value of the hysteretance component changes over time, and a port characteristic for the relationship between the magnetomotive force [symbol] _{C} across the two terminals of the hysteretance component and the magnetic flux Φ_{C} flowing through the hysteretance component is

$${Φ}_{C} = - \frac{d ( {ℱ}_{C} C )}{dt} = - C \frac{d {ℱ}_{C}}{dt} - {ℱ}_{C} \frac{dC}{dt} ;$$

and
if the environmental variables of the magnetic circuit where the hysteretance component is located remain unchanged over time, the hysteretance value of the hysteretance component remains unchanged over time, and the port characteristic for the relationship between the magnetomotive force [symbol] _{C} across the two terminals of the hysteretance component and the magnetic flux Φ_{C} flowing through the hysteretance component is

$${Φ}_{C} = - C \frac{d {ℱ}_{C}}{dt} or {ℱ}_{C} = - \frac{1}{C} \int {Φ}_{C} dt .$$

In a preferred technical solution of the present disclosure, based on the fact that the environmental variables of the magnetic circuit where the hysteretance component is located remain unchanged over time, the hysteretance value of the hysteretance component remains unchanged over time, in a case that the magnetic circuit where the hysteretance component is located is excited by a magnetomotive force with stable sine waves, the port characteristic for the relationship between a phasor [symbol] _{C} of the magnetomotive force [symbol] _{C} across the two terminals of the hysteretance component and a phasor \dot{Φ}_{C} of the magnetic flux Φ_{C} flowing through the hysteretance component is

$${\dot{ℱ}}_{C} = j \frac{1}{ω C} {\dot{Φ}}_{C} ,$$

indicating that a phase of the phasor [symbol] _{C} of the magnetomotive force across the two terminals of the hysteretance component leads a phase of the phasor \dot{Φ}_{C} of the magnetic flux flowing through the hysteretance component, where j represents an imaginary number unit.

In a preferred technical solution of the present disclosure, the hysteretance component has a hindering effect on an alternating magnetic flux in the magnetic circuit where the hysteretance component is located and has no hindering effect on a constant magnetic flux in the magnetic circuit where the hysteretance component is located, a hysteretance reactance corresponding to the hysteretance component is

$${𝒳}_{C} = \frac{1}{ω C} ,$$

the hysteretance reactance [symbol] _{C} being used for describing the hindering magnitude of the hysteretance component on the alternating magnetic flux in the magnetic circuit where the hysteretance component is located, with the unit of A/Wb; in a case that the magnetic circuit where the hysteretance component is located is excited by the magnetomotive force with stable sine waves, according to the hysteretance value

$$C = \frac{μ}{ωsin γ} \frac{A}{h}$$

of the hysteretance component, the hysteretance value C decreases as the angular frequency ω of the magnetic source increases; and according to the hysteretance reactance

$${𝒳}_{C} = \frac{1}{ω C} = \frac{sin γ}{μ} \frac{h}{A} ,$$

the hysteretance reactance [symbol] _{C} is independent of the angular frequency ω of the magnetic source corresponding to the magnetic circuit, and the hysteretance reactance [symbol] _{C} is determined by the length h, the cross-sectional area A, the magnetic conductivity μ, and the magnetic hysteresis angle γ of the hysteretance component.

Correspondingly, the present disclosure also provides an application method for the hysteretance component. This method involves connecting a reluctance component and a magductance component in series. By utilizing the various properties of the hysteretance component, the intensity of magnetic hysteresis in the magnetic circuit can be changed, and the magnitude of the effects caused by the magnetic hysteresis can be controlled, enabling the control of the magnitude of magnetic flux while controlling the phase between magnetic flux and magnetomotive force, thereby enhancing the application efficiency of magnetic circuits.

To solve the above technical problems, the present disclosure employs the following technical solution: an application method for the hysteretance component includes forming a target magnetic circuit by connecting the hysteretance component, a reluctance component, a magductance component, and a magnetic source in series, the target magnetic circuit satisfying Kirchhoff's magnetomotive force law in a vector magnetic circuit theory, i.e.,

$$ℱ = {ℱ}_{ℛ} + {ℱ}_{ℒ} + {ℱ}_{C} = {ℛΦ}_{ℛ} + ℒ \frac{d {Φ}_{ℒ}}{dt} + ( - \frac{1}{C} \int {Φ}_{C} dt ) ,$$

where [symbol] and [symbol] _{C} represent magnetomotive forces across two terminals of the reluctance component, the magductance component, and the hysteretance component, respectively, [symbol] represents a reluctance value of the reluctance component, [symbol] represents a magductance value of the magductance component, [symbol] represents a magnetomotive force of the target magnetic circuit, Φ_{R} represents a magnetic flux flowing through the reluctance component, [symbol] represents a magnetic flux flowing through the magductance component, and [symbol] = [symbol] =Φ_{C}=Φ.

In a preferred technical solution of the present disclosure, in a case that the target magnetic circuit is excited by the magnetomotive force with stable sine waves, the Kirchhoff's magnetomotive force law in the vector magnetic circuit theory is obtained according to a phasor method, i.e.,

$$\dot{ℱ} = ℛ {\dot{Φ}}_{ℛ} + j ω ℒ {\dot{Φ}}_{ℒ} + j \frac{1}{ω C} {\dot{Φ}}_{C} ,$$

where [symbol] represents a phasor of the magnetomotive force of the target magnetic circuit, [symbol] represents a phasor of the magnetic flux flowing through the reluctance component, and [symbol] represents a phasor of the magnetic flux flowing through the magductance component.

In a preferred technical solution of the present disclosure, the target magnetic circuit satisfies the Kirchhoff's magnetic flux law and a theorem of magnetic circuits in the vector magnetic circuit theory.

In a preferred technical solution of the present disclosure, a magnetic impedance of the target magnetic circuit is

$$𝒵 = ℛ + j 𝒳 = ℛ + j ( ωℒ + \frac{1}{ω C} ) , \vert 𝒵 \vert = \sqrt{{ℛ}^{2} + {( ωℒ + \frac{1}{ω C} )}^{2}} ,$$

where the magnetic impedance Z includes a reluctance [symbol] and a magnetic reactance

$$𝒳 = ωℒ + \frac{1}{ω C} ,$$

the magnetic reactance [symbol] includes a magductance reactance [symbol] =ω [symbol] and the hysteretance reactance

$${𝒳}_{C} = \frac{1}{ω C} ,$$

and φ represents a magnetic impedance angle of the target magnetic circuit,

$$φ = \frac{φℒ + \frac{1}{ω C}}{ℛ} .$$

In a preferred technical solution of the present disclosure, for the target magnetic circuit, a preset target magnetic flux phasor \dot{Φ}ₘ and a preset target magnetic impedance angle φₘ of the target magnetic circuit are adjusted according to the following steps:
step A: calculating the reluctance [symbol] in the target magnetic circuit according to the angular frequency ω of the magnetic source of the target magnetic circuit and [symbol] = [symbol] =\dot{Φ}_{C}=\dot{Φ}ₘ and according to the target magnetic circuit satisfying the Kirchhoff's magnetomotive force law

$$\dot{ℱ} = ℛ {\dot{Φ}}_{ℛ} + j ω ℒ {\dot{Φ}}_{ℒ} + j \frac{1}{ω C} {\dot{Φ}}_{C} ,$$

calculating the magnetic reactance [symbol] _{eq} in the target magnetic circuit according to

$${𝒳}_{eq} = ωℒ + \frac{1}{ω C} ,$$

obtaining the hysteretance reactance

$${𝒳}_{C} = \frac{1}{ω C} = {𝒳}_{eq}$$

in the target magnetic circuit by ignoring the magductance [symbol] in the target magnetic circuit, and entering step B;
step B: calculating the hysteretance value C_{eq} in the target magnetic circuit according to the hysteretance reactance

$${𝒳}_{C} = \frac{1}{ω {C}_{eq}}$$

and the angular frequency ω of the magnetic source of the target magnetic circuit, and entering step C;
step C: calculating a target hysteretance value C₂ corresponding to the target magnetic circuit according to the formula φₘ=arctan (1/ωC₂ [symbol] ), obtaining an incremental hysteretance value C₁ corresponding to the target magnetic circuit according to the hysteretance value C_{eq} in the target magnetic circuit and based on C₁=1/(1/C₂−1/C_{eq}), and entering step D; and
step D: adding a hysteretance component satisfying the incremental hysteretance value C₁ to the target magnetic circuit, allowing the target magnetic circuit to satisfy the preset target magnetic flux phasor \dot{Φ}ₘ and the preset target magnetic impedance angle φₘ.

In a preferred technical solution of the present disclosure, in step D, according to the incremental hysteretance value C₁ corresponding to the target magnetic circuit, and based on

$${C}_{1} = \frac{μ}{ω sin γ} \frac{A}{h} ,$$

a hysteretance component with the corresponding length h, cross-sectional A, magnetic conductivity μ, and magnetic hysteresis angle γ is selected and added to the target magnetic circuit, allowing the target magnetic circuit to satisfy the preset target magnetic flux phasor \dot{Φ}ₘ and the preset target magnetic impedance angle φₘ; or
according to the incremental hysteretance value C₁ corresponding to the target magnetic circuit, and based on

$${C}_{1} = - \frac{{{\int {Φ}_{{C}_{1}} dt}^{}}_{}}{{ℱ}_{{C}_{1}}}$$

and the port characteristic for the relationship between a magnetomotive force [symbol] _{C}_{₁} across two terminals of the added hysteretance component and a magnetic flux Φ_{C}_{₁} flowing through the added hysteretance component, a corresponding hysteretance component is selected and added to the target magnetic circuit, allowing the target magnetic circuit to satisfy the preset target magnetic flux phasor \dot{Φ}ₘ and the preset target magnetic impedance angle φₘ.

In a preferred technical solution of the present disclosure, reactive power of the target magnetic circuit is

$$Q = ℛ Φ \frac{d Φ}{dt} ,$$

active power of the target magnetic circuit is

$$𝒫 = {𝒫}_{ℒ} + {𝒫}_{C} = \vert ℒ {( \frac{d Φ}{dt} )}^{2} \vert + \vert ( \frac{1}{C} \int Φ dt ) \frac{d Φ}{dt} \vert ,$$

and apparent power of the target magnetic circuit is S= [symbol] +jQ, where

$${𝒫}_{ℒ} = \vert ℒ {( \frac{d Φ}{dt} )}^{2} \vert$$

represents active loss generated on the magductance component in the target magnetic circuit, corresponding to eddy current loss of the target magnetic circuit; and

$${𝒫}_{C} = \vert ( \frac{1}{C} \int Φ dt ) \frac{d Φ}{dt} \vert$$

represents active loss generated on the hysteretance component in the target magnetic circuit, corresponding to magnetic hysteresis loss of the target magnetic circuit; and
in a case that the target magnetic circuit is excited by the magnetomotive force with stable sine waves, the reactive power of the target magnetic circuit is Q=ω [symbol] ∥\dot{Φ}∥², the active power of the target magnetic circuit is

$$𝒫 = {𝒫}_{ℒ} + {𝒫}_{C} = ω ( ωℒ ) {‖ \dot{Φ} ‖}^{2} + ω ( \frac{1}{ω C} ) {‖ \dot{Φ} ‖}^{2} ,$$

the active power on the magductance component is [symbol] =ω(ω [symbol] )∥\dot{Φ}∥², and the active power on the hysteretance component is

$${𝒫}_{C} = ω ( \frac{1}{ω C} ) {‖ \dot{Φ} ‖}^{2} = ω {𝒳}_{C} {‖ \dot{Φ} ‖}^{2} .$$

The present disclosure provides a hysteretance component and an application method thereof, which adopt the above technical solutions, and have the following technical effects over the prior art.

(1) According to the hysteretance component and the application method thereof designed in the present disclosure, the definition, calculation formula, and port characteristic for the hysteretance component are provided; by increasing or decreasing hysteretance components, from the perspective of magnetic circuits, the intensity of magnetic hysteresis and the effects caused by the magnetic hysteresis such as hysteresis loss and residual magnetism can be estimated and controlled in vector magnetic circuits.

(2) According to the hysteretance component and the application method thereof designed in the present disclosure, in a target magnetic circuit formed by the reluctance component, magductance component, and hysteretance component, the reluctance component represents the constant hindering effect on the magnetic flux, the magductance component represents the hindering effect of eddy current on the alternating magnetic flux, and the hysteretance component represents the hindering effect of the magnetic hysteresis effect on alternating magnetic flux. The magnetic circuit parameters corresponding to these three magnetic circuit components quantitatively characterize the three physical phenomena of magnetization, eddy current and magnetic hysteresis in the magnetic circuit, enabling technicians to selectively change the operating characteristics and vector magnetic quantities of the magnetic circuit by adjusting different magnetic circuit parameters.

(3) According to the hysteretance component and the application method thereof designed in the present disclosure, expressions for the active power and reactive power of the target magnetic circuit are provided. In the target magnetic circuit formed by the reluctance component, the magductance component, and the hysteretance component, the reluctance component corresponds to reactive power, the magductance component corresponds to eddy current loss, and the hysteretance component corresponds to magnetic hysteresis loss. According to the power expressions of the three magnetic circuit components, technicians can selectively adjust the active power and reactive power of the magnetic circuit, ensuring that the capacity of the magnetic circuit satisfies the requirements on practical applications.

### BRIEF DESCRIPTION OF THE DRAWINGS

FIG. 1 is a schematic diagram of a hysteretance component and a target magnetic circuit according to the present disclosure;

FIG. 2 is a schematic diagram showing a plurality of hysteretance components connected in series according to the present disclosure;

FIG. 3 is a schematic diagram showing a plurality of hysteretance components connected in parallel according to the present disclosure;

FIG. 4 is a flowchart for the hysteretance component changing the operating state of the magnetic circuit according to the present disclosure;

FIG. 5 is a diagram showing a simulation model of a ferrite magnetic circuit according to the present disclosure;

FIG. 6 is a waveform diagram showing an initial magnetomotive force and initial magnetic flux of the magnetic circuit according to the present disclosure;

FIG. 7 is a diagram showing an equivalent magnetic circuit of a transformer after adding the hysteretance component according to the present disclosure;

FIG. 8 is a waveform diagram showing the magnetomotive force and magnetic flux of the magnetic circuit after adding the hysteretance component according to the present disclosure; and

FIG. 9 is a diagram comparing hysteresis loops of the magnetic circuit before and after adding the hysteretance component according to the present disclosure.

### DETAILED DESCRIPTION

The embodiments of the present disclosure will be further described in detail by reference to the accompanying drawings of the specification.

A hysteretance component is designed in the present disclosure, which is a magnetic medium connected in series within a magnetic circuit, with the core content involving that, by increasing or decreasing hysteretance components in the magnetic circuit, the intensity of magnetic hysteresis in the magnetic circuit is changed pertinently, thereby changing the operating state of the vector magnetic quantities in the magnetic circuit. For instance, in a case that the magnetomotive force is stable in the magnetic circuit, hysteretance components are added to the magnetic circuit to control the intensity of magnetic hysteresis, thereby changing the magnitude of magnetic flux and a phase angle between magnetomotive force and magnetic flux in the magnetic circuit, allowing the vector state of the magnetic flux in the magnetic circuit to be consistent with that of the target magnetic flux.

In physical, the hysteretance component is a magnetic medium having magnetic hysteresis, denoted by the symbol C. Herein, the Lucida Calligraphy font is used to distinguish the component from electrical circuit components.

The designed hysteretance component has a hysteretance value

$$C = \frac{μ}{ωsin γ} \frac{A}{h} = \frac{κ A}{h}$$

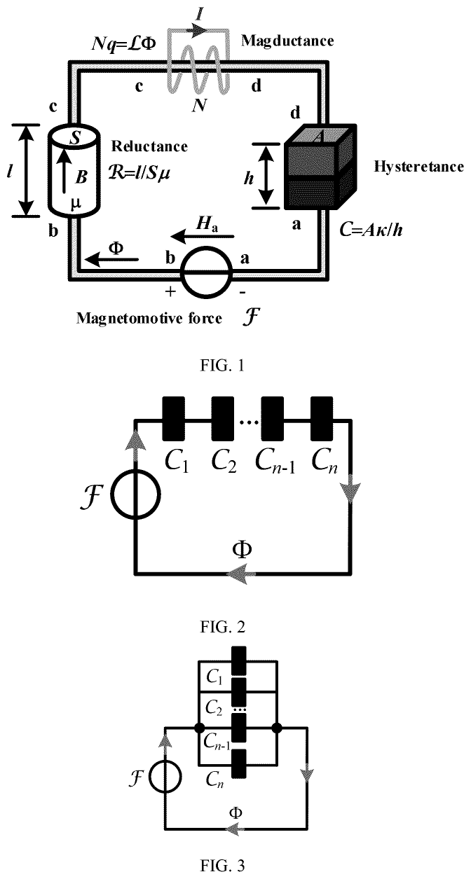

determined based on a length h, a cross-sectional area A, a magnetic conductivity μ, and a magnetic hysteresis angle γ of the hysteretance component, and based on an angular frequency ω of a magnetic source in a magnetic circuit where the hysteretance component is located, as shown in FIG. 1. The unit of the hysteretance can be denoted as Ω·s². The physical meaning of the hysteretance value C is a ratio of an integral (considering the effects caused by demagnetization and magnetization) of a magnetic flux Φ_{C} flowing through the hysteretance component over time to magnetomotive forces [symbol] _{C} across two terminals of the hysteretance component,

$$C = - \frac{\int {Φ}_{{C}^{dt}}}{{ℱ}_{C}} ,$$

i.e. the unit of the hysteretance value is Wb·s/A, where κ represents a magnetic medium coefficient,

$$κ = \frac{μ}{ω sin γ} ,$$

and—represents that a reference direction of the magnetomotive force [symbol] _{C} across the two terminals of the hysteretance component and a reference direction of the magnetic flux Φ_{C} flowing through the hysteretance component are opposite to preset reference directions.

For a structure of a hysteretance component including at least two sub-hysteretance components, in a case that n sub-hysteretance components are connected in series, a hysteretance value of the overall series connection structure is

$$C = 1 / ( \frac{1}{{C}_{1}} + \frac{1}{{C}_{2}} + … \frac{1}{{C}_{n - 1}} + \frac{1}{{C}_{n}} ) ,$$

and in a case that n sub-hysteretance components are connected in parallel, a hysteretance value of the overall parallel connection structure is C=C₁+C₂ . . . +Cₙ₋₁+Cₙ.

The hysteretance component is further designed. If environmental variables of the magnetic circuit where the hysteretance component is located change over time, the hysteretance value of the hysteretance component changes over time, and a port characteristic for the relationship between the magnetomotive force [symbol] _{C} across the two terminals of the hysteretance component and the magnetic flux Φ_{C} flowing through the hysteretance component is

$${Φ}_{C} = - \frac{d ( {ℱ}_{C} C )}{dt} = - C \frac{d {ℱ}_{C}}{dt} - {ℱ}_{C} \frac{dC}{dt} .$$

If the environmental variables of the magnetic circuit where the hysteretance component is located remain unchanged over time, the hysteretance value of the hysteretance component remains unchanged over time, and the port characteristic for the relationship between the magnetomotive force [symbol] _{C} across the two terminals of the hysteretance component and the magnetic flux Φ_{C} flowing through the hysteretance component is

$${Φ}_{C} = - C \frac{d {ℱ}_{C}}{dt} or {ℱ}_{C} = - \frac{1}{C} \int {Φ}_{C} dt .$$

Therefore, based on the fact that the environmental variables of the magnetic circuit where the hysteretance component is located remain unchanged over time and the hysteretance value of the hysteretance component remains unchanged over time, in a case that the magnetic circuit where the hysteretance component is located is excited by a magnetomotive force with stable sine waves, the port characteristic for the relationship between a phasor [symbol] _{C} of the magnetomotive force [symbol] _{C} across the two terminals of the hysteretance component and a phasor \dot{Φ}_{C} of the magnetic flux C flowing through the hysteretance component is

$${\dot{ℱ}}_{C} = j \frac{1}{ω C} {\dot{Φ}}_{C} ,$$

indicating that a phase of the phasor [symbol] _{C} of the magnetomotive force across the two terminals of the hysteretance component leads a phase of the phasor \hat{Φ}_{C} of the magnetic flux flowing through the hysteretance component, where j represents an imaginary number unit.

The hysteretance value of the hysteretance component proposed in the present disclosure can quantitatively represent the magnetic hysteresis loss caused by magnetic hysteresis in magnetic materials under alternating magnetic flux. It also influences the phase between magnetomotive force and the magnetic flux, making the phase of magnetomotive force of the hysteretance component lead the phase of the magnetic flux.

When the hysteretance component is applied to a magnetic circuit, it has a hindering effect on an alternating magnetic flux in the magnetic circuit where the hysteretance component is located and has no hindering effect on a constant magnetic flux in the magnetic circuit where the hysteretance component is located, and a hysteretance reactance corresponding to the hysteretance component is

$${𝒳}_{C} = \frac{1}{ω C} ,$$

the hysteretance reactance [symbol] _{C} being used for describing the hindering magnitude of the hysteretance component on the alternating magnetic flux in the magnetic circuit where the hysteretance component is located, with the unit of A/Wb. In a case that the magnetic circuit where the hysteretance component is located is excited by the magnetomotive force with stable sine waves, according to the hysteretance value

$$C = \frac{μ}{ω sin γ} \frac{A}{h}$$

of the hysteretance component, the hysteretance value C decreases as the angular frequency ω of the magnetic source increases. According to the hysteretance reactance

$${𝒳}_{C} = \frac{1}{ω C} = \frac{sin γ}{μ} \frac{h}{A} ,$$

the hysteretance reactance [symbol] _{C} is independent of the angular frequency ω of the magnetic source corresponding to the magnetic circuit, and remains unchanged. The hysteretance reactance [symbol] _{C} is determined by the length h, the cross-sectional area A, the magnetic conductivity μ, and the magnetic hysteresis angle γ of the hysteretance component.

Based on the above definition, calculation formulas and port characteristics for the hysteretance component, the application method for the hysteretance component is further designed, as shown in FIG. 1. The specific design involves forming a target magnetic circuit by connecting the hysteretance component, a reluctance component, a magductance component, and a magnetic source in series. The target magnetic circuit satisfies Kirchhoff's magnetomotive force law in the vector magnetic circuit theory, Kirchhoff's magnetic flux law in the vector magnetic circuit theory, as well as other magnetic circuit theorems in the vector magnetic circuit theory such as Thevenin theorem, Norton theorem, and substitution theorem.

For the Kirchhoff's magnetomotive force law in the vector magnetic circuit theory, i.e., magnetomotive force of the target magnetic circuit

$$ℱ = {ℱ}_{ℛ} + {ℱ}_{L} + {ℱ}_{C} = ℛ {Φ}_{ℛ} + ℒ \frac{d {Φ}_{ℒ}}{dt} + ( - \frac{1}{C} \int {Φ}_{C} dt ) ,$$

where [symbol] and [symbol] _{C} represent magnetomotive forces across two terminals of the reluctance component, the magductance component, and the hysteretance component, respectively, [symbol] represents a reluctance value of the reluctance component, [symbol] represents a magductance value of the magductance component, [symbol] represents a magnetic flux flowing through the reluctance component, and [symbol] represents a magnetic flux flowing through the magductance component. Due to the series connection among the hysteretance component, the reluctance component, and the magductance component, [symbol] = [symbol] =Φ_{C}=Φ.

As shown in FIG. 1, in a case that the target magnetic circuit is excited by the magnetomotive force with stable sine waves, the Kirchhoff's magnetomotive force law in the vector magnetic circuit theory is obtained according to a phasor method, i.e., a phasor of the magnetomotive force of the target magnetic circuit

$$\dot{ℱ} = ℛ {\dot{Φ}}_{ℛ} + j ω ℒ {\dot{Φ}}_{ℒ} + j \frac{1}{ω C} {\dot{Φ}}_{C} ,$$

where [symbol] represents a phasor of the magnetic flux flowing through the reluctance component, and [symbol] represents a phasor of the magnetic flux flowing through the magductance component.

When the designed target magnetic circuit is applied in practice, a magnetic impedance in the target magnetic circuit is involved. The magnetic impedance

$$𝒵 = ℛ + j 𝒳 = ℛ + j ( ω ℒ + \frac{1}{ω C} ) , \vert 𝒵 \vert = \sqrt{{ℛ}^{2} + {( ωℒ + \frac{1}{ω C} )}^{2}} .$$

Specifically, the magnetic impedance Z includes a reluctance [symbol] and a magnetic reactance

$$𝒳 = ω ℒ + \frac{1}{ω C} .$$

Furthermore, the magnetic reactance [symbol] includes a magductance reactance [symbol] =ω [symbol] and the hysteretance reactance

$${𝒳}_{C} = \frac{1}{ω C} .$$

and φ represents a magnetic impedance angle of the target magnetic circuit,

$$φ = \frac{φ ℒ + \frac{1}{ω C}}{ℛ} .$$

Based on the foregoing design of the hysteretance component and the application thereof in the target magnetic circuit, in practical applications, the hysteretance value of the hysteretance component is adjusted by selecting the length h, the cross-sectional area A, the magnetic conductivity μ, and the magnetic hysteresis angle γ of a magnetic medium. Alternatively, the hysteretance value of the hysteretance component is adjusted according to the physical meaning of the hysteretance value C that represents the ratio of the integral of the magnetic flux Φ_{C} flowing through the hysteretance component over time t to the magnetomotive force [symbol] _{C} across two terminals of the hysteretance component and according to the port characteristic for the relationship between the magnetomotive force [symbol] _{C} across the two terminals of the hysteretance component and the magnetic flux flowing through the hysteretance component. Therefore, the intensity of the magnetic hysteresis in the target magnetic circuit can be adjusted, thereby changing the amplitude and phase of the magnetic flux in the magnetic circuit.

Furthermore, for the design of the hysteretance component in the target magnetic circuit, hysteretance components can be added or reduced in the magnetic circuit to alter the intensity of the magnetic hysteresis in the target magnetic circuit, thereby controlling the magnitude of effects caused by the magnetic hysteresis, enabling the vector state of magnetic flux in the target magnetic circuit to be consistent with that of the target magnetic flux.

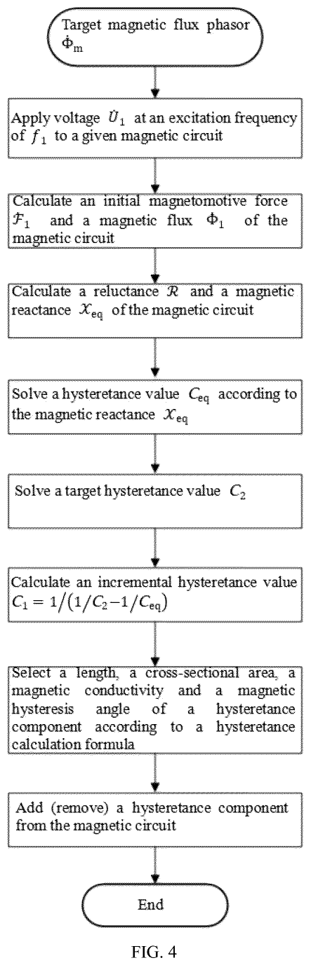

In practical applications, specifically for the target magnetic circuit, as shown in FIG. 4, a preset target magnetic flux phasor \dot{Φ}ₘ and a preset target magnetic impedance angle om of the target magnetic circuit are adjusted according to the following steps A-D.

Step A: the reluctance [symbol] in the target magnetic circuit is calculated according to the angular frequency ω of the magnetic source of the target magnetic circuit and [symbol] = [symbol] =\dot{Φ}_{C}=\dot{Φ}ₘ and according to the target magnetic circuit satisfying the Kirchhoff's magnetomotive force law

$$\dot{ℱ} = ℛ {\dot{Φ}}_{ℛ} + j ω ℒ {\dot{Φ}}_{ℒ} + j \frac{1}{ω C} {\dot{Φ}}_{C} ;$$

the magnetic reactance [symbol] _{eq} in the target magnetic circuit is calculated according to

$${𝒳}_{eq} = ω ℒ + \frac{1}{ω C} ;$$

the hysteretance reactance

$${𝒳}_{C} = \frac{1}{ω C} = {𝒳}_{eq}$$

in the target magnetic circuit is obtained by ignoring the magductance [symbol] in the target magnetic circuit; and step B is entered.

Step B: the hysteretance value C_{eq} in the target magnetic circuit is calculated according to the hysteretance reactance

$${𝒳}_{C} = \frac{1}{ω {C}_{eq}}$$

and the angular frequency ω of the magnetic source of the target magnetic circuit, and step C is entered.

Step C: a target hysteretance value C₂ corresponding to the target magnetic circuit is calculated according to the formula φₘ=arctan (1/ωC₂ [symbol] ); an incremental hysteretance value C₁ corresponding to the target magnetic circuit is obtained according to the hysteretance value C_{eq} in the target magnetic circuit and based on C₁=1/(1/C₂−1/C_{eq}); and step D is entered.

Step D: a hysteretance component satisfying the incremental hysteretance value C₁ is added to the target magnetic circuit, allowing the target magnetic circuit to satisfy the preset target magnetic flux phasor \dot{Φ}ₘ and the preset target magnetic impedance angle φₘ.

According to the design of the hysteretance component, step D is designed with two implementation modes in practical application. One mode involves that, according to the incremental hysteretance value C₁ corresponding to the target magnetic circuit and

$${C}_{1} = \frac{μ}{ω sin γ} \frac{A}{h} ,$$

a hysteretance component with the corresponding length h, cross-sectional area A, magnetic conductivity μ, and magnetic hysteresis angle γ is selected and added to the target magnetic circuit, allowing the target magnetic circuit to satisfy the preset target magnetic flux phasor \dot{Φ}ₘ and the preset target magnetic impedance angle φₘ.

The other mode involves that, according to the incremental hysteretance value C₁ corresponding to the target magnetic circuit,

$${C}_{1} = - \frac{\int {Φ}_{{C}_{1}} dt}{{ℱ}_{{C}_{1}}} ,$$

and the port characteristic for the relationship between a magnetomotive force [symbol] _{C} across two terminals of the added hysteretance component and a magnetic flux Φ_{C}_{₁} flowing through the added hysteretance component, a corresponding hysteretance component is selected and added to the target magnetic circuit, allowing the target magnetic circuit to satisfy the preset target magnetic flux phasor \dot{Φ}ₘ and the preset target magnetic impedance angle φₘ.

The port characteristic during the implementation of the second mode is

$${Φ}_{C} = - C \frac{d {ℱ}_{C}}{dt} , or {ℱ}_{C} = - \frac{1}{C} \int {Φ}_{C} dt ,$$

referring to the relationship between the magnetomotive force [symbol] _{C} across the two terminals of the applied hysteretance component and the magnetic flux Φ_{C} flowing through the applied hysteretance component in a case that the environmental variables in the magnetic circuit where the hysteretance component is located remain unchanged over time and the hysteretance value of the hysteretance component also remains unchanged over time.

In practical applications, upon determining a hysteretance component satisfying the incremental hysteretance value C₁ added to the magnetic circuit, if this hysteretance component is formed by a plurality of sub-hysteretance components connected in parallel or in series, it is necessary to consider the hysteretance values of each sub-hysteretance component under the series or parallel connection structure. In a case that n sub-hysteretance components are connected in series, the hysteretance value of the overall series connection structure is

$${C}_{eq} = 1 / ( \frac{1}{{C}_{1}} + \frac{1}{{C}_{2}} + … \frac{1}{{C}_{n - 1}} + \frac{1}{{C}_{n}} ) ,$$

as shown in FIG. 2. In a case that n sub-hysteretance components are connected in parallel, the hysteretance value of the overall parallel connection structure is C_{eq}=C₁+C₂ . . . Cₙ₋₁+Cₙ, as shown in FIG. 3.

Therefore, after the hysteretance component satisfying the incremental hysteretance value C₁ to be added to the target magnetic circuit is determined, based on the calculation method for the hysteretance value under the aforementioned series and parallel connection structures, the composition of sub-hysteretance components corresponding to the incremental hysteretance value C₁ under different connection relationships can be calculated and determined. The composition can be further applied in the target magnetic circuit, enabling the target magnetic circuit to satisfy the preset target magnetic flux phasor \dot{Φ}ₘ and the preset target magnetic impedance angle φₘ.

In actual applications, based on the target magnetic circuit under the application of the designed hysteretance circuit of the present disclosure, reactive power of the target magnetic circuit is

$$Q = ℛ Φ \frac{d Φ}{dt} ,$$

active power of the target magnetic circuit is

$$𝒫 = {𝒫}_{ℒ} + {𝒫}_{C} = \vert ℒ {( \frac{d Φ}{dt} )}^{2} \vert + \vert ( \frac{1}{C} \int Φ dt ) \frac{d Φ}{dt} \vert ,$$

and apparent power of the target magnetic circuit is S=P+jQ, where

$${𝒫}_{ℒ} = \vert ℒ {( \frac{d Φ}{dt} )}^{2} \vert$$

represents active loss generated on the magductance component in the target magnetic circuit, corresponding to eddy current loss of the target magnetic circuit; and

$${𝒫}_{C} = \vert ( \frac{1}{C} \int Φ dt ) \frac{d Φ}{dt} \vert$$

represents active loss generated on the hysteretance component in the target magnetic circuit, corresponding to magnetic hysteresis loss of the target magnetic circuit.

In a further application, in a case that the target magnetic circuit is excited by the magnetomotive force with stable sine waves, the reactive power of the target magnetic circuit is Q=ω [symbol] ∥\dot{Φ}∥², the active power of the target magnetic circuit is

$$𝒫 = {𝒫}_{ℒ} + {𝒫}_{C} = ω ( ωℒ ) {‖ \dot{Φ} ‖}^{2} + ω ( \frac{1}{ω C} ) {‖ \dot{Φ} ‖}^{2} ,$$

the active power on the magductance component is P_{L}=ω(ωL)∥\dot{Φ}∥², and the active power on the hysteretance component is

$${𝒫}_{C} = ω ( \frac{1}{ω C} ) {‖ \dot{Φ} ‖}^{2} = {ω𝒳}_{C} {‖ \dot{Φ} ‖}^{2} .$$

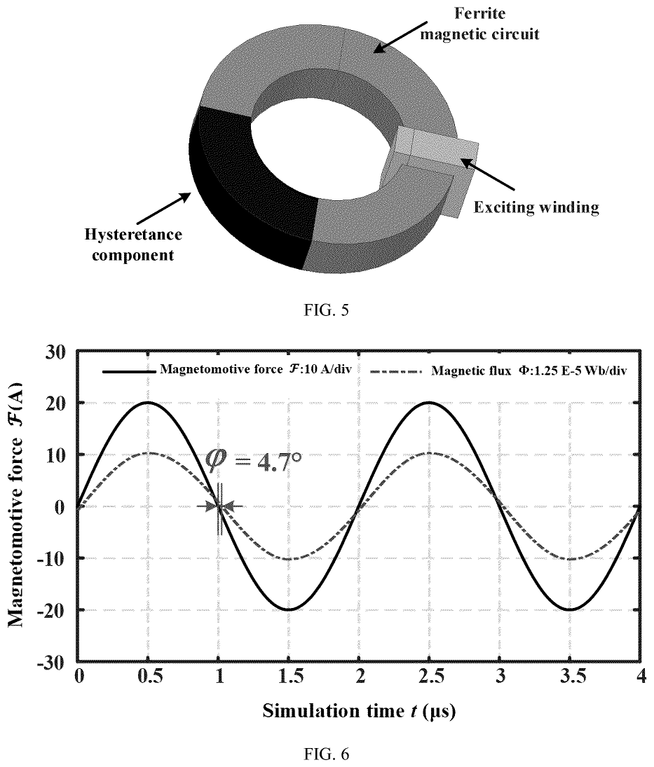

The design scheme described above is implemented in actual, and validated using ANSYS simulation software. A closed ferrite magnetic circuit with an inner diameter of 9.6 mm, an outer diameter of 16.0 mm, and a height of 6.3 mm is constructed in ANSYS, as shown in FIG. 5. The eddy current loss in the magnetic circuit is set as 0, which corresponds to a magductance parameter [symbol] of 0. The target magnetic flux amplitude Φₘ is set as 1.26×10⁻⁵ Wb, and the target magnetic impedance angle om is set as 12°. The initial magnetic circuit as shown in FIG. 6 is transformed into the target magnetic circuit as shown in FIG. 8 by adding hysteretance components in the magnetic circuit, with the flowchart shown in FIG. 4. Firstly, the amplitude of the magnetomotive force [symbol] ₁ is set as 20 A, with a frequency of f=500 kHz. When the magnetic circuit operates stably, the waveforms of the magnetomotive force [symbol] ₁ and the magnetic flux Φ₁ are shown in FIG. 8. The equivalent reluctance value [symbol] _{eq}=1554163.724H⁻¹ and the equivalent magnetic reactance value [symbol] _{eq}=127775.5173A/Wb are calculated according to the Kirchhoff's magnetomotive force law

$$\dot{ℱ} = R \dot{Φ} + j \frac{1}{ω C} Φ ,$$

and an initial magnetic impedance angle of φ=4.7° can be obtained. The target hysteretance value C₂=9.525 Wb·s/A is obtained according to the target magnetic impedance angle φₘ=12° and the formula φₘ=arctan (1/ωC₂ [symbol] _{eq}). Therefore, the hysteretance value added to the magnetic circuit is C₁=1/(1/C₂−1/C_{eq})=15.55 Wb·s/A.

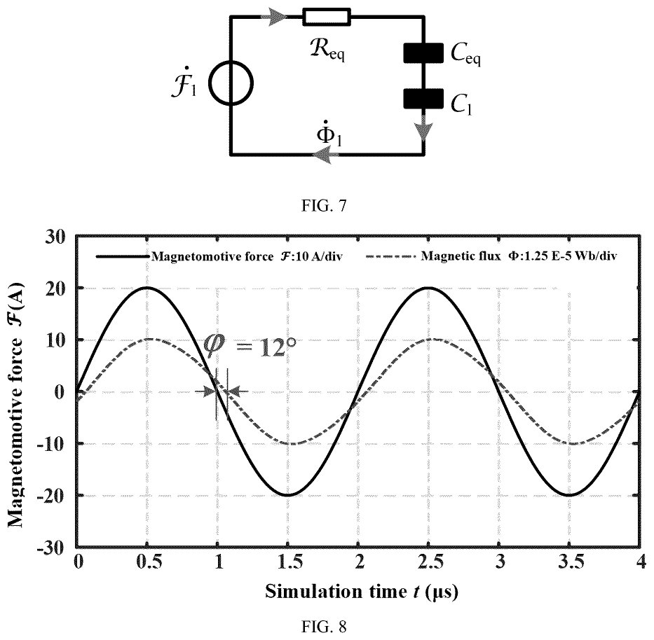

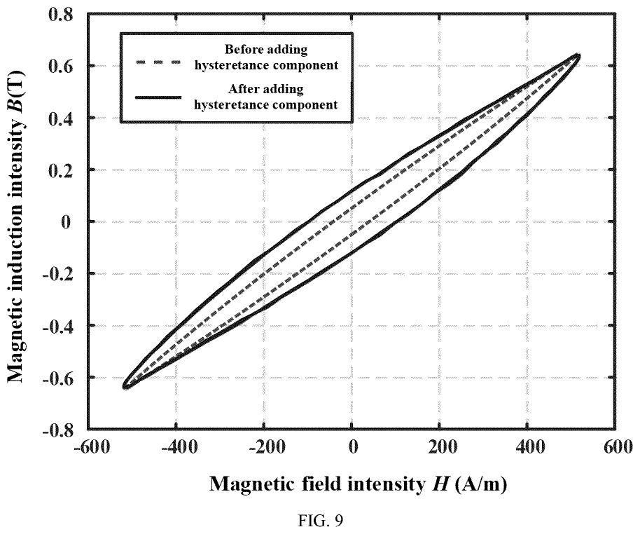

Through the combination of the length h, cross-sectional area A, the magnetic conductivity μ, and the magnetic hysteresis angle γ of the magnetic medium, multiple sets of eligible magnetic media can be obtained. The equivalent magnetic circuit after adding the hysteretance component is shown in FIG. 7. Keeping the amplitude of the magnetomotive force [symbol] ₁ at 20 A, after adding the hysteretance component, the waveforms of the magnetomotive force [symbol] ₁ and the magnetic flux Φ₁ in the magnetic circuit of a transformer are shown in FIG. 7. It can be seen that the magnetic impedance angle φ of the magnetic circuit of transformer reaches the target magnetic impedance angle φₘ, and the magnetic flux Φ₁ reaches the target magnetic flux amplitude Φₘ. Additionally, a comparison of hysteresis loops before and after adding the hysteretance component is shown in FIG. 9. By adding the hysteretance component, the area of the hysteresis loop increases, and the magnetic hysteresis in the magnetic circuit is enhanced, verifying the feasibility of controlling the intensity of the magnetic hysteresis by hysteretance components.

According to the hysteretance component and the application method thereof designed in the aforementioned technical solution, the definition, calculation formula, and port characteristics for the hysteretance component are provided. By adding or subtracting hysteretance components, from the perspective of the magnetic circuit, it is possible to estimate and control the intensity of the magnetic hysteresis and the effects caused by the magnetic hysteresis such as magnetic hysteresis loss and remanence effect.

In the target magnetic circuit formed by the reluctance component, the magductance component, and the hysteretance component, the reluctance component represents the constant hindering effect on the magnetic flux, the magductance component represents the hindering effect of eddy current on the alternating magnetic flux, and the hysteretance component represents the hindering effect of the magnetic hysteresis on the alternating magnetic flux. The magnetic circuit parameters corresponding to these three magnetic circuit components quantitatively characterize the three physical phenomena of magnetization, eddy current, and magnetic hysteresis in the magnetic circuit, enabling technicians to selectively change the operating characteristics and the vector magnetic quantities of the magnetic circuit by adjusting different magnetic circuit parameters.

Furthermore, for the target magnetic circuit, expressions for the active power and reactive power of the target magnetic circuit are provided. In the target magnetic circuit formed by the reluctance component, the magductance component, and the hysteretance component, the reluctance component corresponds to the reactive power, the magductance component corresponds to the eddy current loss, and the hysteretance component corresponds to the magnetic hysteresis loss. According to the power expressions of the three magnetic circuit components, technicians can selectively adjust the active power and reactive power of the magnetic circuit, ensuring that the capacity of the magnetic circuit satisfies requirements on practical application.

The embodiments of the present disclosure are described in detail above by reference to the accompanying drawings, but the present disclosure is not limited to this. Various changes can be made within the scope of knowledge possessed by those ordinary skilled in the art without departing from the principle of the present disclosure.

1. A hysteretance component, having a hysteretance value
$$C = \frac{μ}{ω sin γ} \frac{A}{h} = \frac{κ A}{h}$$
determined based on a length h, a cross-sectional area A, a magnetic conductivity μ, and a magnetic hysteresis angle γ of the hysteretance component, and based on an angular frequency ω of a magnetic source in a magnetic circuit where the hysteretance component is located, with the unit of Wb·s/A=Ω·s², wherein the physical meaning of the hysteretance value C is a ratio of an integral of a magnetic flux Φ_{C} flowing through the hysteretance component over time to magnetomotive forces [symbol] _{C} across two terminals of the hysteretance component, i.e.,
$$C = - \frac{\int {Φ}_{{C}^{dt}}}{{ℱ}_{C}} ,$$
κ representing a magnetic medium coefficient,
$$κ = \frac{μ}{ω sin γ} ,$$
and—representing that a reference direction of the magnetomotive force [symbol] _{C} across the two terminals of the hysteretance component and a reference direction of the magnetic flux Φ_{C} flowing through the hysteretance component are opposite to preset reference directions; and
for a structure of a hysteretance component comprising at least two sub-hysteretance components, in a case that n sub-hysteretance components are connected in series, the hysteretance value of the overall series connection structure is
$$C = 1 / ( \frac{1}{{C}_{1}} + \frac{1}{{C}_{2}} + … \frac{1}{{C}_{n - 1}} + \frac{1}{{C}_{n}} ) ,$$
and in a case that n sub-hysteretance components are connected in parallel, the hysteretance value of the overall parallel connection structure is C=C₁+C₂ . . . +Cₙ₋₁+Cₙ.

2. The hysteretance component according to claim 1, wherein if environmental variables of the magnetic circuit where the hysteretance component is located change over time, the hysteretance value of the hysteretance component changes over time, and a port characteristic for the relationship between the magnetomotive force [symbol] _{C} across the two terminals of the hysteretance component and the magnetic flux Φ_{C} flowing through the hysteretance component is
$${Φ}_{C} = - \frac{d ( {ℱ}_{C} C )}{dt} = - C \frac{d {ℱ}_{C}}{dt} - {ℱ}_{C} \frac{dC}{dt} ;$$
and
if the environmental variables of the magnetic circuit where the hysteretance component is located remain unchanged over time, the hysteretance value of the hysteretance component remains unchanged over time, and the port characteristic for the relationship between the magnetomotive force [symbol] _{C} across the two terminals of the hysteretance component and the magnetic flux Φ_{C} flowing through the hysteretance component is
$${Φ}_{C} = - C \frac{d {ℱ}_{C}}{dt} or {ℱ}_{C} = - \frac{1}{C} \int {Φ}_{C} dt .$$

3. The hysteretance component according to claim 2, wherein based on the fact that the environmental variables of the magnetic circuit where the hysteretance component is located remain unchanged over time and the hysteretance value of the hysteretance component remains unchanged over time, in a case that the magnetic circuit where the hysteretance component is located is excited by a magnetomotive force with stable sine waves, the port characteristic for the relationship between a phasor [symbol] _{C} of the magnetomotive force [symbol] _{C} across the two terminals of the hysteretance component and a phasor \dot{Φ}_{C} of the magnetic flux Φ_{C} flowing through the hysteretance component is
$${\dot{ℱ}}_{C} = j \frac{1}{ω C} {\dot{Φ}}_{C} ,$$
indicating that a phase of the phasor [symbol] _{C} of the magnetomotive force across the two terminals of the hysteretance component leads a phase of the phasor \dot{Φ}_{C} of the magnetic flux flowing through the hysteretance component, where j represents an imaginary number unit.

4. The hysteretance component according to claim 1, wherein the hysteretance component has a hindering effect on an alternating magnetic flux in the magnetic circuit where the hysteretance component is located and has no hindering effect on a constant magnetic flux in the magnetic circuit where the hysteretance component is located, a hysteretance reactance corresponding to the hysteretance component is
$${𝒳}_{C} = \frac{1}{ω C} ,$$
the hysteretance reactance [symbol] _{C} being used for describing the hindering magnitude of the hysteretance component on the alternating magnetic flux in the magnetic circuit where the hysteretance component is located, with the unit of A/Wb; in a case that the magnetic circuit where the hysteretance component is located is excited by the magnetomotive force with stable sine waves, according to the hysteretance value
$$C = \frac{μ}{ω sin γ} \frac{A}{h}$$
of the hysteretance component, the hysteretance value C decreases as the angular frequency ω of the magnetic source increases; and according to the hysteretance reactance
$${𝒳}_{C} = \frac{1}{ω C} = \frac{sin γ}{μ} \frac{h}{A} ,$$
the hysteretance reactance [symbol] _{C} is independent of the angular frequency ω of the magnetic source corresponding to the magnetic circuit, and the hysteretance reactance [symbol] _{C} is determined by the length h, the cross-sectional area A, the magnetic conductivity μ, and the magnetic hysteresis angle γ of the hysteretance component.

5. An application method for the hysteretance component according to claim 1, comprising forming a target magnetic circuit by connecting the hysteretance component, a reluctance component, a magductance component, and a magnetic source in series, the target magnetic circuit satisfying Kirchhoff's magnetomotive force law in a vector magnetic circuit theory, i.e.,
$$ℱ = {ℱ}_{ℛ} + {ℱ}_{ℒ} + {ℱ}_{C} = {ℛΦ}_{ℛ} + ℒ \frac{d {Φ}_{ℒ}}{dt} + ( - \frac{1}{C} \int {Φ}_{C} d t ) ,$$
where [symbol] and [symbol] _{C} represent magnetomotive forces across two terminals of the reluctance component, the magductance component, and the hysteretance component, respectively, [symbol] represents a reluctance value of the reluctance component, [symbol] represents a magductance value of the magductance component, [symbol] represents a magnetomotive force of the target magnetic circuit, [symbol] represents a magnetic flux flowing through the reluctance component, [symbol] represents a magnetic flux flowing through the magductance component, and [symbol] = [symbol] =Φ_{C}=Φ.

6. The application method for the hysteretance component according to claim 5, wherein in a case that the target magnetic circuit is excited by the magnetomotive force with stable sine waves, the Kirchhoff's magnetomotive force law in the vector magnetic circuit theory is obtained according to a phasor method, i.e.,
$$\dot{ℱ} = ℛ {\dot{Φ}}_{ℛ} + j ωℒ {\dot{Φ}}_{ℒ} + j \frac{1}{ω C} {\dot{Φ}}_{C} ,$$
where [symbol] represents a phasor of the magnetomotive force of the target magnetic circuit, [symbol] represents a phasor of the magnetic flux flowing through the reluctance component, and [symbol] represents a phasor of the magnetic flux flowing through the magductance component.

7. The application method for the hysteretance component according to claim 5, wherein the target magnetic circuit satisfies the Kirchhoff's magnetic flux law and a theorem of magnetic circuits in the vector magnetic circuit theory.

8. The application method for the hysteretance component according to claim 5, wherein a magnetic impedance of the target magnetic circuit is
$$𝒵 = ℛ + j 𝒳 = ℛ + j ( ωℒ + \frac{1}{ω C} ) , \vert 𝒵 \vert = \sqrt{{ℛ}^{2} + {( ωℒ + \frac{1}{ω C} )}^{2}} ,$$
where the magnetic impedance Z comprises the reluctance [symbol] and a magnetic reactance
$$𝒳 = ωℒ + \frac{1}{ω C} ,$$
the magnetic reactance [symbol] comprises a magductance reactance [symbol] =ω [symbol] and the hysteretance reactance
$${𝒳}_{C} = \frac{1}{ω C} ,$$
and φ represents a magnetic impedance angle of the target magnetic circuit,
$$φ = \frac{φℒ + \frac{1}{ω C}}{ℛ} .$$

9. The application method for the hysteretance component according to claim 5, wherein for the target magnetic circuit, a preset target magnetic flux phasor \dot{Φ}ₘ and a preset target magnetic impedance angle φₘ of the target magnetic circuit are adjusted according to the following steps:
step A: calculating the reluctance [symbol] in the target magnetic circuit according to the angular frequency ω of the magnetic source of the target magnetic circuit and [symbol] = [symbol] =\dot{Φ}_{C}=Φₘ and according to the target magnetic circuit satisfying the Kirchhoff's magnetomotive force law
$$\dot{ℱ} = ℛ {\dot{Φ}}_{ℛ} + j ωℒ {\dot{Φ}}_{ℒ} + j \frac{1}{ω C} {\dot{Φ}}_{C} ,$$
calculating the magnetic reactance [symbol] _{eq} in the target magnetic circuit according to
$${𝒳}_{e q} = ωℒ + \frac{1}{ω C} ,$$
obtaining the hysteretance reactance
$${𝒳}_{C} = \frac{1}{ω C} = {𝒳}_{e q}$$
in the target magnetic circuit by ignoring the magductance [symbol] in the target magnetic circuit, and entering step B;
step B: calculating the hysteretance value C_{eq} in the target magnetic circuit according to the hysteretance reactance
$${𝒳}_{C} = \frac{1}{ω {C}_{e q}}$$
and the angular frequency ω of the magnetic source of the target magnetic circuit, and entering step C;
step C: calculating a target hysteretance value C₂ corresponding to the target magnetic circuit according to the formula Φₘ=arctan(1/ωC₂ [symbol] ), obtaining an incremental hysteretance value C₁ corresponding to the target magnetic circuit according to the hysteretance value C_{eq} in the target magnetic circuit and based on C₁=1/(1/C₂−1/C_{eq}), and entering step D; and
step D: adding a hysteretance component satisfying the incremental hysteretance value C₁ to the target magnetic circuit, allowing the target magnetic circuit to satisfy the preset target magnetic flux phasor \dot{Φ}ₘ and the preset target magnetic impedance angle φₘ.

10. The application method for the hysteretance component according to claim 9, wherein in step D, according to the incremental hysteretance value C₁ corresponding to the target magnetic circuit and based on
$${C}_{1} = \frac{μ}{ω sin γ} \frac{A}{h} ,$$
a hysteretance component with the corresponding length h, cross-sectional area A, magnetic conductivity μ, and magnetic hysteresis angle γ is selected and added to the target magnetic circuit, allowing the target magnetic circuit to satisfy the preset target magnetic flux phasor \dot{Φ}ₘ and the preset target magnetic impedance angle φₘ; or
according to the incremental hysteretance value C₁ corresponding to the target magnetic circuit and based on
$${C}_{1} = - \frac{\int {Φ}_{{C}_{1}} dt}{{ℱ}_{{C}_{1}}}$$
and the port characteristic for the relationship between a magnetomotive force [symbol] _{C}_{₁} across two terminals of the added hysteretance component and a magnetic flux Φ_{C}_{₁} flowing through the added hysteretance component, a corresponding hysteretance component is selected and added to the target magnetic circuit, allowing the target magnetic circuit to satisfy the preset target magnetic flux phasor \dot{Φ}ₘ and the preset target magnetic impedance angle φₘ.

11. The application method for the hysteretance component according to claim 9, wherein reactive power of the target magnetic circuit is
$$Q = ℛΦ \frac{d Φ}{dt} ,$$
active power of the target magnetic circuit is
$$𝒫 = {𝒫}_{ℒ} + {𝒫}_{C} = \vert {ℒ ( \frac{d Φ}{dt} )}^{2} \vert + \vert ( \frac{1}{C} \int Φ d t ) \frac{d Φ}{dt} \vert ,$$
and apparent power of the target magnetic circuit is S= [symbol] +jQ, where
$${𝒫}_{ℒ} = \vert {ℒ ( \frac{d Φ}{dt} )}^{2} \vert$$
represents active loss generated on the magductance component in the target magnetic circuit, corresponding to eddy current loss of the target magnetic circuit; and
$${𝒫}_{C} = \vert ( \frac{1}{C} \int Φ d t ) \frac{d Φ}{dt} \vert$$
represents active loss generated on the hysteretance component in the target magnetic circuit, corresponding to magnetic hysteresis loss of the target magnetic circuit; and
in a case that the target magnetic circuit is excited by the magnetomotive force with stable sine waves, the reactive power of the target magnetic circuit is Q=ω [symbol] ∥\hat{Φ}∥², the active power of the target magnetic circuit is
$$𝒫 = {𝒫}_{ℒ} + {𝒫}_{C} = ω ( ωℒ ) {‖ \dot{Φ} ‖}^{2} + ω ( \frac{1}{ω C} ) {‖ \dot{Φ} ‖}^{2} ,$$
the active power on the magductance component is [symbol] =ω(ω [symbol] )∥\dot{Φ}∥², and the active power on the hysteretance component is
$${𝒫}_{C} = ω ( \frac{1}{ω C} ) {‖ \dot{Φ} ‖}^{2} = {ω𝒳}_{C} {‖ \dot{Φ} ‖}^{2} .$$

12. An application method for the hysteretance component according to claim 2, comprising forming a target magnetic circuit by connecting the hysteretance component, a reluctance component, a magductance component, and a magnetic source in series, the target magnetic circuit satisfying Kirchhoff's magnetomotive force law in a vector magnetic circuit theory, i.e.,
$$ℱ = {ℱ}_{ℛ} + {ℱ}_{ℒ} + {ℱ}_{C} = {ℛΦ}_{ℛ} + ℒ \frac{d {Φ}_{ℒ}}{dt} + ( - \frac{1}{C} \int {Φ}_{C} d t ) ,$$
where [symbol] and [symbol] _{C} represent magnetomotive forces across two terminals of the reluctance component, the magductance component, and the hysteretance component, respectively, [symbol] represents a reluctance value of the reluctance component, [symbol] represents a magductance value of the magductance component, [symbol] represents a magnetomotive force of the target magnetic circuit, [symbol] represents a magnetic flux flowing through the reluctance component, [symbol] represents a magnetic flux flowing through the magductance component, and [symbol] = [symbol] =Φ_{C}=Φ.

13. An application method for the hysteretance component according to claim 3, comprising forming a target magnetic circuit by connecting the hysteretance component, a reluctance component, a magductance component, and a magnetic source in series, the target magnetic circuit satisfying Kirchhoff's magnetomotive force law in a vector magnetic circuit theory, i.e.,
$$ℱ = {ℱ}_{ℛ} + {ℱ}_{ℒ} + {ℱ}_{C} = {ℛΦ}_{ℛ} + ℒ \frac{d {Φ}_{ℒ}}{dt} + ( - \frac{1}{C} \int {Φ}_{C} d t ) ,$$
where [symbol] and [symbol] _{C} represent magnetomotive forces across two terminals of the reluctance component, the magductance component, and the hysteretance component, respectively, [symbol] represents a reluctance value of the reluctance component, [symbol] represents a magductance value of the magductance component, [symbol] represents a magnetomotive force of the target magnetic circuit, [symbol] represents a magnetic flux flowing through the reluctance component, [symbol] represents a magnetic flux flowing through the magductance component, and [symbol] = [symbol] =Φ_{C}=Φ.

14. An application method for the hysteretance component according to claim 4, comprising forming a target magnetic circuit by connecting the hysteretance component, a reluctance component, a magductance component, and a magnetic source in series, the target magnetic circuit satisfying Kirchhoff's magnetomotive force law in a vector magnetic circuit theory, i.e.,
$$ℱ = {ℱ}_{ℛ} + {ℱ}_{ℒ} + {ℱ}_{C} = {ℛΦ}_{ℛ} + ℒ \frac{d {Φ}_{ℒ}}{dt} + ( - \frac{1}{C} \int {Φ}_{C} d t ) ,$$
where [symbol] and [symbol] _{C} represent magnetomotive forces across two terminals of the reluctance component, the magductance component, and the hysteretance component, respectively, [symbol] represents a reluctance value of the reluctance component, [symbol] represents a magductance value of the magductance component, [symbol] represents a magnetomotive force of the target magnetic circuit, [symbol] represents a magnetic flux flowing through the reluctance component, [symbol] represents a magnetic flux flowing through the magductance component, and [symbol] = [symbol] =Φ_{C}=Φ.

15. The application method for the hysteretance component according to claim 6, wherein for the target magnetic circuit, a preset target magnetic flux phasor \dot{Φ}ₘ and a preset target magnetic impedance angle φₘ of the target magnetic circuit are adjusted according to the following steps:
step A: calculating the reluctance [symbol] in the target magnetic circuit according to the angular frequency ω of the magnetic source of the target magnetic circuit and [symbol] = [symbol] =\dot{Φ}_{C}=\dot{Φ}ₘ and according to the target magnetic circuit satisfying the Kirchhoff's magnetomotive force law
$$\dot{ℱ} = ℛ {\dot{Φ}}_{ℛ} + j ω ℒ {\dot{Φ}}_{ℒ} + j \frac{1}{ω C} {\dot{Φ}}_{C} ,$$
calculating the magnetic reactance [symbol] _{eq} in the target magnetic circuit according to
$${𝒳}_{e q} = ωℒ + \frac{1}{ω C} ,$$
obtaining the hysteretance reactance
$${𝒳}_{C} = \frac{1}{ω C} = {𝒳}_{e q}$$
in the target magnetic circuit by ignoring the magductance [symbol] in the target magnetic circuit, and entering step B;
step B: calculating the hysteretance value C_{eq} in the target magnetic circuit according to the hysteretance reactance
$${𝒳}_{C} = \frac{1}{ω {C}_{eq}}$$
and the angular frequency ω of the magnetic source of the target magnetic circuit, and entering step C;
step C: calculating a target hysteretance value C₂ corresponding to the target magnetic circuit according to the formula φₘ=arctan (1/ωC₂ [symbol] ), obtaining an incremental hysteretance value C₁ corresponding to the target magnetic circuit according to the hysteretance value C_{eq} in the target magnetic circuit and based on C₁=1/(1/C₂−1/C_{eq}), and entering step D; and
step D: adding a hysteretance component satisfying the incremental hysteretance value C₁ to the target magnetic circuit, allowing the target magnetic circuit to satisfy the preset target magnetic flux phasor \dot{Φ}ₘ and the preset target magnetic impedance angle φₘ.

16. The application method for the hysteretance component according to claim 7, wherein for the target magnetic circuit, a preset target magnetic flux phasor \dot{Φ}ₘ and a preset target magnetic impedance angle φₘ of the target magnetic circuit are adjusted according to the following steps:
step A: calculating the reluctance [symbol] in the target magnetic circuit according to the angular frequency ω of the magnetic source of the target magnetic circuit and [symbol] = [symbol] =\dot{Φ}_{C}=\dot{Φ}ₘ and according to the target magnetic circuit satisfying the Kirchhoff's magnetomotive force law
$$\dot{ℱ} = ℛ {\dot{Φ}}_{ℛ} + j ω ℒ {\dot{Φ}}_{ℒ} + j \frac{1}{ω C} {\dot{Φ}}_{C} ,$$
calculating the magnetic reactance [symbol] _{eq} in the target magnetic circuit according to
$${𝒳}_{eq} = ω ℒ + \frac{1}{ω C} ,$$
obtaining the hysteretance reactance
$${𝒳}_{C} = \frac{1}{ω C} = {𝒳}_{eq}$$
in the target magnetic circuit by ignoring the magductance [symbol] in the target magnetic circuit, and entering step B;
step B: calculating the hysteretance value C_{eq} in the target magnetic circuit according to the hysteretance reactance
$${𝒳}_{C} = \frac{1}{ω {C}_{eq}}$$
and the angular frequency ω of the magnetic source of the target magnetic circuit, and entering step C;
step C: calculating a target hysteretance value C₂ corresponding to the target magnetic circuit according to the formula φₘ=arctan(1/ωC₂ [symbol] ), obtaining an incremental hysteretance value C₁ corresponding to the target magnetic circuit according to the hysteretance value C_{eq} in the target magnetic circuit and based on C₁=1/(1/C₂−1/C_{eq}), and entering step D; and
step D: adding a hysteretance component satisfying the incremental hysteretance value C₁ to the target magnetic circuit, allowing the target magnetic circuit to satisfy the preset target magnetic flux phasor \dot{Φ}ₘ and the preset target magnetic impedance angle φₘ.

17. The application method for the hysteretance component according to claim 8, wherein for the target magnetic circuit, a preset target magnetic flux phasor \dot{Φ}ₘ and a preset target magnetic impedance angle φₘ of the target magnetic circuit are adjusted according to the following steps:
step A: calculating the reluctance [symbol] in the target magnetic circuit according to the angular frequency ω of the magnetic source of the target magnetic circuit and [symbol] = [symbol] =\dot{Φ}_{C}=\dot{Φ}ₘ and according to the target magnetic circuit satisfying the Kirchhoff's magnetomotive force law
$$\dot{ℱ} = ℛ {\dot{Φ}}_{ℛ} + j ω ℒ {\dot{Φ}}_{ℒ} + j \frac{1}{ω C} {\dot{Φ}}_{C} ,$$
calculating the magnetic reactance [symbol] _{eq} in the target magnetic circuit according to
$${𝒳}_{eq} = ω ℒ + \frac{1}{ω C} ,$$
obtaining the hysteretance reactance
$${𝒳}_{C} = \frac{1}{ω C} = {𝒳}_{eq}$$
in the target magnetic circuit by ignoring the magductance [symbol] in the target magnetic circuit, and entering step B;
step B: calculating the hysteretance value C_{eq} in the target magnetic circuit according to the hysteretance reactance
$${𝒳}_{C} = \frac{1}{ω {C}_{eq}}$$
and the angular frequency ω of the magnetic source of the target magnetic circuit, and entering step C;
step C: calculating a target hysteretance value C₂ corresponding to the target magnetic circuit according to the formula φₘ=arctan(1/ωC₂ [symbol] ), obtaining an incremental hysteretance value C₁ corresponding to the target magnetic circuit according to the hysteretance value C_{eq} in the target magnetic circuit and based on C₁=1/(1/C₂−1/C_{eq}), and entering step D; and
step D: adding a hysteretance component satisfying the incremental hysteretance value C₁ to the target magnetic circuit, allowing the target magnetic circuit to satisfy the preset target magnetic flux phasor \dot{Φ}ₘ and the preset target magnetic impedance angle φₘ.

18. The application method for the hysteretance component according to claim 15, wherein in step D, according to the incremental hysteretance value C₁ corresponding to the target magnetic circuit and based on
$${C}_{1} = \frac{μ}{ω sin γ} \frac{A}{h} ,$$
a hysteretance component with the corresponding length h, cross-sectional area A, magnetic conductivity μ, and magnetic hysteresis angle γ is selected and added to the target magnetic circuit, allowing the target magnetic circuit to satisfy the preset target magnetic flux phasor \dot{Φ}ₘ and the preset target magnetic impedance angle φₘ; or
according to the incremental hysteretance value C₁ corresponding to the target magnetic circuit and based on
$${C}_{1} = - \frac{\int {Φ}_{{C}_{1}} dt}{{ℱ}_{{C}_{1}}}$$
and the port characteristic for the relationship between a magnetomotive force [symbol] _{C}_{₁} across two terminals of the added hysteretance component and a magnetic flux c, flowing through the added hysteretance component, a corresponding hysteretance component is selected and added to the target magnetic circuit, allowing the target magnetic circuit to satisfy the preset target magnetic flux phasor \dot{Φ}ₘ and the preset target magnetic impedance angle φₘ.

19. The application method for the hysteretance component according to claim 16, wherein in step D, according to the incremental hysteretance value C₁ corresponding to the target magnetic circuit and based on
$${C}_{1} = \frac{μ}{ω sin γ} \frac{A}{h} ,$$
a hysteretance component with the corresponding length h, cross-sectional area A, magnetic conductivity μ, and magnetic hysteresis angle γ is selected and added to the target magnetic circuit, allowing the target magnetic circuit to satisfy the preset target magnetic flux phasor \dot{Φ}ₘ and the preset target magnetic impedance angle φₘ; or
according to the incremental hysteretance value C₁ corresponding to the target magnetic circuit and based on
$${C}_{1} = - \frac{\int {Φ}_{{C}_{1}} dt}{{ℱ}_{{C}_{1}}}$$
and the port characteristic for the relationship between a magnetomotive force [symbol] _{C}_{₁}, across two terminals of the added hysteretance component and a magnetic flux Φ_{C}_{₁} flowing through the added hysteretance component, a corresponding hysteretance component is selected and added to the target magnetic circuit, allowing the target magnetic circuit to satisfy the preset target magnetic flux phasor \dot{Φ}ₘ and the preset target magnetic impedance angle φₘ.

20. The application method for the hysteretance component according to claim 17, wherein in step D, according to the incremental hysteretance value C₁ corresponding to the target magnetic circuit and based on
$${C}_{1} = \frac{μ}{ω sin γ} \frac{A}{h} ,$$
a hysteretance component with the corresponding length h, cross-sectional area A, magnetic conductivity μ, and magnetic hysteresis angle γ is selected and added to the target magnetic circuit, allowing the target magnetic circuit to satisfy the preset target magnetic flux phasor \dot{Φ}ₘ and the preset target magnetic impedance angle φₘ; or
according to the incremental hysteretance value C₁ corresponding to the target magnetic circuit and based on
$${C}_{1} = - \frac{\int {Φ}_{{C}_{1}} dt}{{ℱ}_{{C}_{1}}}$$
and the port characteristic for the relationship between a magnetomotive force [symbol] _{C}_{₁} across two terminals of the added hysteretance component and a magnetic flux dc, flowing through the added hysteretance component, a corresponding hysteretance component is selected and added to the target magnetic circuit, allowing the target magnetic circuit to satisfy the preset target magnetic flux phasor \dot{Φ}ₘ and the preset target magnetic impedance angle φₘ.
